(19) United States
(12) Patent Application Publication
(10) Pub. No.: US 2011/0257709 A1
(43) Pub. Date: Oct. 20, 2011

(54) METHOD AND APPARATUS FOR MODIFYING NEUROSTIMULATION LINEAR LEAD SHAPE TO CORRECT LEAD MIGRATION

(75) Inventors: Douglas Michael Ackermann, Palo Alto, CA (US); Anne Margaret Pianca, Santa Monica, CA (US); Michael Adam Moffitt, Valencia, CA (US)

(73) Assignee: BOSTON SCIENTIFIC NEUROMODULATION CORPORATION, Valencia, CA (US)

(21) Appl. No.: 13/090,810

(22) Filed: Apr. 20, 2011

Related U.S. Application Data
(60) Provisional application No. 61/326,131, filed on Apr. 20, 2010.

(51) Int. Cl. A61N 1/05; A61N 1/36

(52) U.S. Cl. 607/62; 607/116

(57) ABSTRACT

A neurostimulation system comprises an implantable neurostimulation lead, an implantable neurostimulator configured for delivering stimulation energy to the implantable neurostimulation lead, an actuating device configured for modifying a linear shape of the lead after it has migrated from a baseline position, memory configured for storing a threshold value, and a processor configured for determining a magnitude at which the lead has migrated from the baseline position, comparing the determined magnitude to the threshold value, and prompting the actuating device to modify the linear shape of the lead based on the comparison. A method of correcting the migration of a neurostimulation lead implanted within the patient comprises determining a magnitude at which the implanted lead has migrated from a baseline position, comparing the determined magnitude to a threshold value, and modifying the linear shape of the lead based on the comparison.

### RELATED APPLICATION

The present application claims the benefit under 35 U.S.C. §119 to U.S. provisional patent application Ser. No. 61/326,131, filed Apr. 20, 2010. The foregoing application is hereby incorporated by reference into the present application in its entirety.

### FIELD OF THE INVENTION

The present invention relates to tissue stimulation systems, and more particularly, to apparatus and methods for determining migration of neurostimulation leads.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, Functional Electrical Stimulation (FES) systems such as the Freehand system by NeuroControl (Cleveland, Ohio) have been applied to restore some functionality to paralyzed extremities in spinal cord injury patients. Furthermore, in recent investigations Peripheral Nerve Stimulation (PNS) systems have demonstrated efficacy in the treatment of chronic pain syndromes and incontinence, and a number of additional applications are currently under investigation. Occipital Nerve Stimulation (ONS), in which leads are implanted in the tissue over the occipital nerves, has shown promise as a treatment for various headaches, including migraine headaches, cluster headaches, and cervicogenic headaches.

These implantable neurostimulation systems typically include one or more electrode carrying stimulation leads, which are implanted at the desired stimulation site, and a neurostimulator (e.g., an implantable pulse generator (IPG)) implanted remotely from the stimulation site, but coupled either directly to the stimulation lead(s) or indirectly to the stimulation lead(s) via a lead extension. Thus, electrical pulses can be delivered from the neurostimulator to the stimulation leads to stimulate the tissue and provide the desired efficacious therapy to the patient. The neurostimulation system may further comprise a handheld patient programmer in the form of a remote control (RC) to remotely instruct the neurostimulator to generate electrical stimulation pulses in accordance with selected stimulation parameters. A typical stimulation parameter set may include the electrodes that are acting as anodes or cathodes, as well as the amplitude, duration, and rate of the stimulation pulses. The RC may, itself, be programmed by a clinician, for example, by using a clinician's programmer (CP), which typically includes a general purpose computer, such as a laptop, with a programming software package installed thereon. Typically, the RC can only control the neurostimulator in a limited manner (e.g., by only selecting a program or adjusting the pulse amplitude or pulse width), whereas the CP can be used to control all of the stimulation parameters, including which electrodes are cathodes or anodes.

In the context of an SCS procedure, one or more stimulation leads are introduced through the patient's back into the epidural space, such that the electrodes carried by the leads are arranged in a desired pattern and spacing to create an electrode array. One type of commercially available stimulation leads is a percutaneous lead, which comprises a cylindrical body with ring electrodes, and can be introduced into contact with the affected spinal tissue through a Touhy-like needle, which passes through the skin, between the desired vertebrae, and into the epidural space above the dura layer. For unilateral pain, a percutaneous lead is placed on the corresponding lateral side of the spinal cord. For bilateral pain, a percutaneous lead is placed down the midline of the spinal cord, or two or more percutaneous leads are placed down the respective sides of the midline of the spinal cord, and if a third lead is used, down the midline of the special cord. After proper placement of the stimulation leads at the target area of the spinal cord, the leads are anchored in place at an exit site to prevent movement of the stimulation leads. To facilitate the location of the neurostimulator away from the exit point of the stimulation leads, lead extensions are sometimes used.

The stimulation leads, or the lead extensions, are then connected to the IPG, which can then be operated to generate electrical pulses that are delivered, through the electrodes, to the targeted tissue, and in particular, the dorsal column and dorsal root fibers within the spinal cord. The stimulation creates the sensation known as paresthesia, which can be characterized as an alternative sensation that replaces the pain signals sensed by the patient. Intra-operatively (i.e., during the surgical procedure), the neurostimulator may be operated to test the effect of stimulation and adjust the parameters of the stimulation for optimal pain relief. The patient may provide verbal feedback regarding the presence of paresthesia over the pain area, and based on this feedback, the lead positions may be adjusted and re-anchored if necessary. A computer program, such as Bionic Navigator®, available from Boston Scientific Neuromodulation Corporation, can be incorporated in a clinician's programmer (CP) (briefly discussed above) to facilitate selection of the stimulation parameters. Any incisions are then closed to fully implant the system. Post-operatively (i.e., after the surgical procedure has been completed), a clinician can adjust the stimulation parameters using the computerized programming system to re-optimize the therapy.

The efficacy of SCS is related to the ability to stimulate the spinal cord tissue corresponding to evoked paresthesia in the region of the body where the patient experiences pain. Thus, the working clinical paradigm is that achievement of an effective result from SCS depends on the neurostimulation lead or leads being placed in a location (both longitudinal and lateral) relative to the spinal tissue such that the electrical stimulation will induce paresthesia located in approximately the same place in the patient's body as the pain (i.e., the target of treatment). If a lead is not correctly positioned, it is possible that the patient will receive little or no benefit from an implanted SCS system. Thus, correct lead placement can mean the difference between effective and ineffective pain therapy, and as such, precise positioning of the leads proximal to the targets of stimulation is critical to the success of the therapy.

Although the lead(s) may initially be correctly positioned relative to the stimulation target(s), the lead(s) are at risk of migration relative to each other and/or relative to the stimulation target(s). As a result, the therapy provided to the patient by the neurostimulation system may be compromised. Once this occurs, the patient may have to schedule another visit to the physician or clinician in order to adjust the stimulation parameters of the system by reprogramming the neurostimulator to compensate for the lead migration. Until the neurostimulator is reprogrammed, however, the patient will not be getting the quality of therapy previously provided by the neurostimulation system. Furthermore, before realizing that a visit to the physician or clinician is necessary, the patient may attempt to improve the compromised therapy by adjusting the stimulation energy delivered by the neurostimulation system via operation of the RC. However, not knowing that the lead migration is the reason for the compromised therapy, and given that the RC only has limited control over the neurostimulator (which typically allows only selection of programs and adjustment of pulse amplitude and pulse width), the patient will not be able to compensate for lead migration, which typically would require a modification in the electrodes that serve as cathodes/anodes—a skill a patient would typically not have.

There, thus, remains a technique that better addresses the needs of a user when an implanted stimulation lead has migrated in the patient.

### SUMMARY OF THE INVENTION

In accordance with one aspect of the present inventions, a neurostimulation system is provided. The neurostimulation system comprises an implantable neurostimulation lead, an implantable neurostimulator configured for delivering stimulation energy to the implantable neurostimulation lead, and an actuating device configured for modifying a linear shape of the neurostimulation lead after the implanted neurostimulation lead has migrated from a baseline position. The baseline position may be, e.g., a position of the neurostimulation lead relative to tissue or a position of the neurostimulation lead relative to another implantable neurostimulation lead.

In one embodiment, the actuating mechanism comprises at least one steering wire coupled to a distal end of the neurostimulation lead, wherein the steering wire(s) is configured for being tensioned to laterally deflect the distal end of the neurostimulation lead. In another embodiment, the actuating mechanism comprises a fluid-filled bladder extending through the neurostimulation lead, wherein the pressure within the fluid-filled bladder is configured for being increased to straighten the distal end of the neurostimulation lead. In still another embodiment, the actuating mechanism comprises a plurality of a cylindrical segments spaced apart along the neurostimulation lead, wherein the cylindrical segments are configured to be displaced in contact with each other to straighten the distal end of the neurostimulation lead.

The neurostimulation system further comprises at least one processor configured for determining a magnitude at which the neurostimulation lead has migrated from the baseline position. In one embodiment, the processor(s) is configured for determining the magnitude at which the implanted neurostimulation lead has migrated by determining a current position of the implanted neurostimulation lead and computing a difference between the current position and the baseline position. To determine the current position of the implanted neurostimulation lead, the neurostimulation may be configured for transmitting an electrical signal between one or more electrodes carried by the implanted neurostimulation lead and one or more other electrodes, and measuring an electrical parameter in response to the transmission of the electrical signal.

The neurostimulation system further comprises memory configured for storing a threshold value (e.g., representing an acceptable lead position tolerance), and the processor(s) is further configured for comparing the determined magnitude to the threshold value, and prompting the actuating device to modify the linear shape of the neurostimulation lead based on the comparison of the determined magnitude to the threshold value. In one embodiment, the processor(s) is configured for prompting the actuating device to modify the linear shape of the neurostimulation lead (which may be performed automatically) only if the measured relative position is equal to or exceeds the threshold value. The processor(s) may be, e.g., carried by the neurostimulator, or may be carried by an external device, in which case, the processor(s) may be configured for prompting the actuating device to modify the linear shape of the neurostimulation lead upon operative connection of the external device and the neurostimulator.

In accordance with another aspect of the present inventions, a method of correcting the migration of a neurostimulation lead implanted within a patient is provided. The method comprises determining a magnitude at which the implanted neurostimulation lead has migrated from a baseline position, which may be, e.g., the position at which the neurostimulation lead was initially implanted in the patient, and as discussed above, may be, e.g., a position of the neurostimulation lead relative to tissue or a position of the neurostimulation lead relative to another neurostimulation lead implantable within the patient. The magnitude at which the implanted neurostimulation lead had migrated from the baseline position may be accomplished in the same manner described above.

The method further comprises comparing the determined magnitude to a threshold value (e.g., representing an acceptable lead position tolerance), and modifying the linear shape of the neurostimulation lead (e.g., by laterally deflecting the distal end of the neurostimulation lead or straightening the distal end of the neurostimulation lead) based on the comparison of the determined magnitude to the threshold value (e.g., automatically only if the determined magnitude is equal to or exceeds the threshold value). In one method, the linear shape of the neurostimulation lead is modified upon operative connection between a neurostimulator connected to the neurostimulation lead and an external device.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is plan view of one embodiment of a spinal cord stimulation (SCS) system arranged in accordance with the present inventions;

FIG. 2 is a plan view of an implantable pulse generator (IPG) and another embodiment of a percutaneous stimulation lead used in the SCS system of FIG. 1;

FIG. 2A is a cross-sectional view of one percutaneous stimulation lead used in the SCS system of FIG. 1;

FIG. 3 is a plan view of the SCS system of FIG. 1 in use with a patient;

FIG. 4 is a block diagram of the internal components of the IPG of FIG. 1;

FIG. 5 is a plan view of a remote control that can be used in the SCS system of FIG. 1;

FIG. 6 is a block diagram of the internal componentry of the remote control of FIG. 5;

FIG. 7 is a block diagram of the components of a clinician's programmer that can be used in the SCS system of FIG. 1;

FIG. 8A is a plan view of two percutaneous neurostimulation leads implanted along a spinal cord, wherein one of the leads has laterally migrated away from a midline of the spinal cord;

FIG. 8B is a plan view of the implanted percutaneous neurostimulation leads of FIG. 8A, wherein the linear shape of the laterally migrated lead has been modified to displace the distal end of the lead back towards the midline of the spinal cord;

FIG. 9 a partially cutaway view, longitudinal-sectional view of the neurostimulation lead employing one embodiment of an actuator for modifying the linear shape of the neurostimulation lead;

FIG. 10 is a cross-sectional view of the neurostimulation lead of FIG. 9, taken along the line 10-10;

FIG. 11A is a partially, cut-away plan view of the neurostimulation lead of FIG. 9, wherein the distal end of the lead has laterally migrated away from a midline;

FIG. 11B is a partially, cut-away plan view of the neurostimulation lead of FIG. 9, wherein the distal end of the lead has been laterally deflected towards the midline;

FIG. 12 a partially cutaway view, longitudinal-sectional view of the neurostimulation lead employing another embodiment of an actuator for modifying the linear shape of the neurostimulation lead;

FIG. 13 is a cross-sectional view of the neurostimulation lead of FIG. 12, taken along the line 13-13;

FIG. 14A is a partially, cut-away plan view of the neurostimulation lead of FIG. 12, wherein the distal end of the lead has laterally migrated away from a midline;

FIG. 14B is a partially, cut-away plan view of the neurostimulation lead of FIG. 12, wherein the distal end of the lead has been laterally deflected towards the midline;

FIG. 15 a partially cutaway view, longitudinal-sectional view of the neurostimulation lead employing still another embodiment of an actuator for modifying the linear shape of the neurostimulation lead;

FIG. 16 is a cross-sectional view of the neurostimulation lead of FIG. 15, taken along the line 16-16;

FIG. 17A is a partially, cut-away plan view of the neurostimulation lead of FIG. 15, wherein the distal end of the lead has laterally migrated away from a midline; and

FIG. 17B is a partially, cut-away plan view of the neurostimulation lead of FIG. 15, wherein the distal end of the lead has been laterally deflected towards the midline.

### DETAILED DESCRIPTION OF THE EMBODIMENTS

The description that follows relates to a spinal cord stimulation (SCS) system. However, it is to be understood that while the invention lends itself well to applications in SCS, the invention, in its broadest aspects, may not be so limited. Rather, the invention may be used with any type of implantable electrical circuitry used to stimulate tissue. For example, the present invention may be used as part of a multi-lead system such as a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neural stimulator configured to treat urinary incontinence, sleep apnea, shoulder sublaxation, headache, etc.

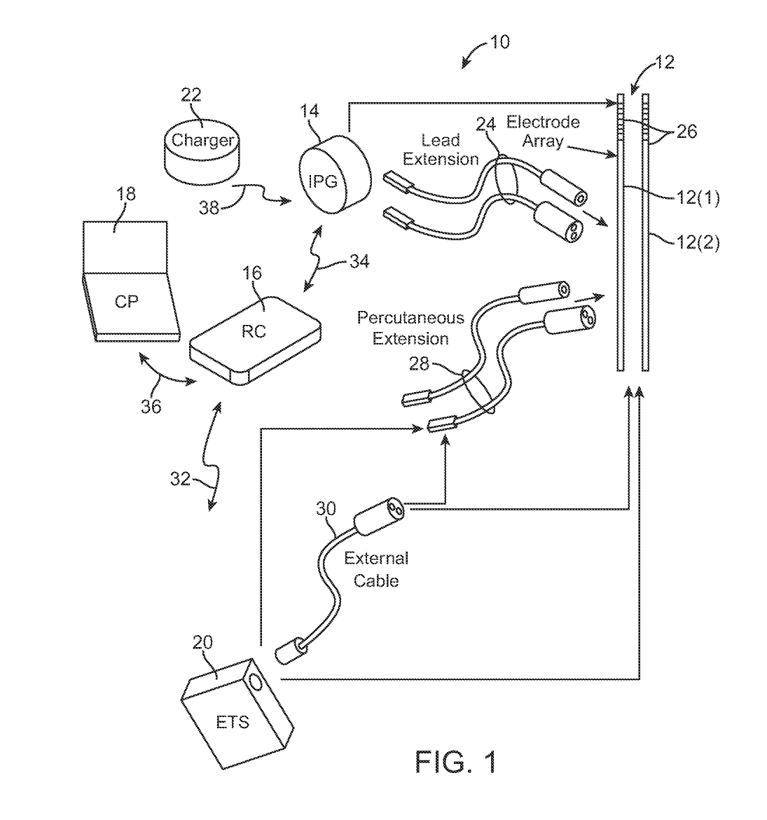

Turning first to FIG. 1, an exemplary SCS system 10 generally comprises a plurality of neurostimulation leads 12 (in this case, two percutaneous leads 12(1) and 12(2)), an implantable pulse generator (IPG) 14, an external remote control (RC) 16, a Clinician's Programmer (CP) 18, an External Trial Stimulator (ETS) 20, and an external charger 22.

The IPG 14 is physically connected via two lead extensions 24 to the neurostimulation leads 12, which carry a plurality of electrodes 26 arranged in an array. As will also be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters. The IPG 14 and neurostimulation leads 12 can be provided as an implantable neurostimulation kit, along with, e.g., a hollow needle, a stylet, a tunneling tool, and a tunneling straw. Further details discussing implantable kits are disclosed in U.S. Application Ser. No. 61/030,506, entitled “Temporary Neurostimulation Lead Identification Device,” which is expressly incorporated herein by reference.

The ETS 20 may also be physically connected via percutaneous lead extensions 28 or external cable 30 to the neurostimulation lead 12. The ETS 20, which has similar pulse generation circuitry as the IPG 14, also delivers electrical stimulation energy in the form of a pulse electrical waveform to the electrode array 26 in accordance with a set of stimulation parameters. The major difference between the ETS 20 and the IPG 14 is that the ETS 20 is a non-implantable device that is used on a trial basis after the neurostimulation lead 12 has been implanted and prior to implantation of the IPG 14, to test the responsiveness of the stimulation that is to be provided. Further details of an exemplary ETS are described in U.S. Pat. No. 6,895,280, which is expressly incorporated herein by reference.

The RC 16 may be used to telemetrically control the ETS 20 via a bi-directional RF communications link 32. Once the IPG 14 and stimulation lead 12 is implanted, the RC 16 may be used to telemetrically control the IPG 14 via a bi-directional RF communications link 34. Such control allows the IPG 14 to be turned on or off and to be programmed with different stimulation programs after implantation. Once the IPG 14 has been programmed, and its power source has been charged or otherwise replenished, the IPG 14 may function as programmed without the RC 16 being present.

The CP 18 provides clinician detailed stimulation parameters for programming the IPG 14 and ETS 20 in the operating room and in follow-up sessions. The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown).

The external charger 22 is a portable device used to transcutaneously charge the IPG 14 via an inductive link 38. For purposes of brevity, the details of the external charger 22 will not be described herein. Details of exemplary embodiments of external chargers are disclosed in U.S. Pat. No. 6,895,280, which has been previously incorporated herein by reference. Once the IPG 14 has been programmed, and its power source has been charged by the external charger 22 or otherwise replenished, the IPG 14 may function as programmed without the RC 16 or CP 18 being present.

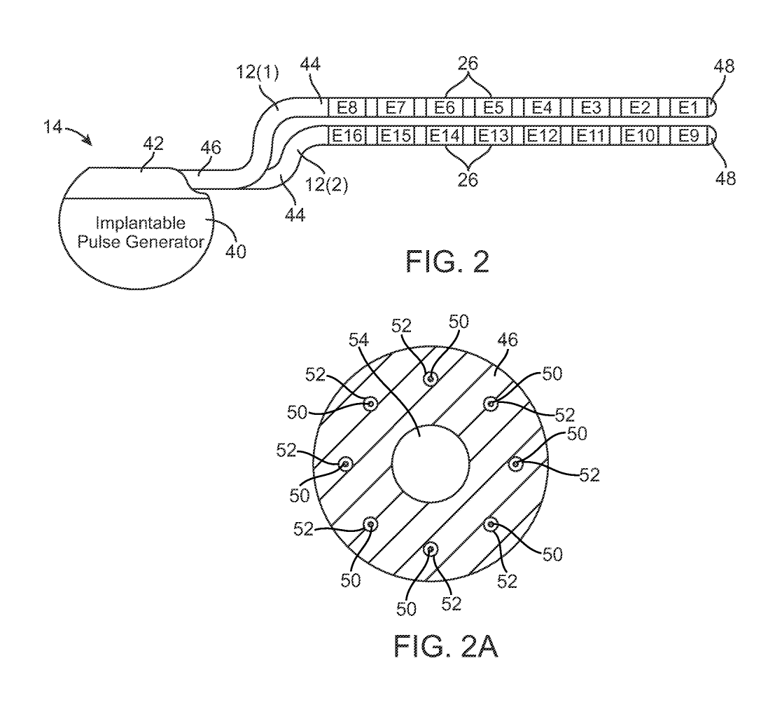

Referring now to FIG. 2, the external features of the neurostimulation leads 12 and the IPG 14 will be briefly described.

The IPG 14 comprises an outer case 40 for housing the electronic and other components (described in further detail below). The outer case 40 is composed of an electrically conductive, biocompatible material, such as titanium, and forms a hermetically sealed compartment wherein the internal electronics are protected from the body tissue and fluids. In some cases, the outer case 40 may serve as an electrode. The IPG 14 further comprises a connector 42 to which the proximal ends of the neurostimulation leads 12 mate in a manner that electrically couples the electrodes 26 to the internal electronics (described in further detail below) within the outer case 40. To this end, the connector 42 includes two ports (not shown) for receiving the proximal ends of the two percutaneous leads 12. In the case where the lead extensions 24 are used, the ports may instead receive the proximal ends of such lead extensions 24.

As will be described in further detail below, the IPG 14 includes pulse generation circuitry that provides electrical stimulation energy to the electrodes 26 in accordance with a set of parameters. Such parameters may comprise electrode combinations, which define the electrodes that are activated as anodes (positive), cathodes (negative), and turned off (zero), and electrical pulse parameters, which define the pulse amplitude (measured in milliamps or volts depending on whether the IPG 14 supplies constant current or constant voltage to the electrodes), pulse duration (measured in microseconds), pulse rate (measured in pulses per second), and pulse shape.

With respect to the pulse patterns provided during operation of the SCS system 10, electrodes that are selected to transmit or receive electrical energy are referred to herein as “activated,” while electrodes that are not selected to transmit or receive electrical energy are referred to herein as “non-activated.” Electrical energy delivery will occur between two (or more) electrodes, one of which may be the IPG case 40, so that the electrical current has a path from the energy source contained within the IPG case 40 to the tissue and a sink path from the tissue to the energy source contained within the case. Electrical energy may be transmitted to the tissue in a monopolar or multipolar (e.g., bipolar, tripolar, etc.) fashion.

Monopolar delivery occurs when a selected one or more of the lead electrodes 26 is activated along with the case 40 of the IPG 14, so that electrical energy is transmitted between the selected electrode 26 and case 40. Monopolar delivery may also occur when one or more of the lead electrodes 26 are activated along with a large group of lead electrodes located remotely from the one or more lead electrodes 26 so as to create a monopolar effect; that is, electrical energy is conveyed from the one or more lead electrodes 26 in a relatively isotropic manner. Bipolar delivery occurs when two of the lead electrodes 26 are activated as anode and cathode, so that electrical energy is transmitted between the selected electrodes 26. Tripolar delivery occurs when three of the lead electrodes 26 are activated, two as anodes and the remaining one as a cathode, or two as cathodes and the remaining one as an anode.

Each neurostimulation lead 12 includes an elongated lead body 44 having a proximal end 46 and a distal end 48. The lead body 44 may, e.g., have a diameter within the range of 0.03 inches to 0.07 inches and a length within the range of 10 cm to 90 cm for spinal cord stimulation applications. The lead body 44 may be composed of a suitable electrically insulative material, such as, a polymer (e.g., polyurethane or silicone), and may be extruded from as a unibody construction.

Each neurostimulation lead 12 further comprises a plurality of terminals (not shown) mounted to the proximal end 46 of the lead body 44 and the plurality of in-line electrodes 26 (in this case, eight electrodes E1-E8 for the neurostimulation lead 12(1) and eight electrodes E9-E16 for the neurostimulation lead 12(2)) mounted to the distal end 48 of the lead body 44. Although each neurostimulation lead 12 is shown as having eight electrodes 26 (and thus, eight corresponding terminals), the number of electrodes may be any number suitable for the application in which the neurostimulation lead 12 is intended to be used (e.g., two, four, sixteen, etc.). Each of the electrodes 26 takes the form of a cylindrical ring element composed of an electrically conductive, non-corrosive, material, such as, e.g., platinum, platinum iridium, titanium, or stainless steel, which is circumferentially disposed about the lead body 44.

As shown in FIG. 2A, each neurostimulation lead 12 also includes a plurality of electrical conductors 50 extending through individual lumens 52 within the lead body 44 and connected between the respective terminals (not shown) and electrodes 26 using suitable means, such as welding, thereby electrically coupling the proximally-located terminals with the distally-located electrodes 26. In the illustrated embodiment, each conductor 50 is a multfilar cable (1×19 or 1×7) wire made from 28% inner core of pure silver with 72% outer cladding of MP35N stainless steel (although other materials may be used such as MP with a Pt core, pure MP, pure Pt, MP with different percentage Ag inner core). Each conductor 50 is then insulated with a thin outer jacket (0.001″ thick) of Ethylene Tetrafluoroethylene (ETFE) fluoro-based polymer (other insulative jacketing materials may be used such as PFA, FEP). In the illustrated embodiment, the conductors 50 can be pre-cut and two zones on the ETFE insulation pre-ablated where they are connected between the respective electrode 26 and terminal. The stimulation lead 14 further includes a central lumen 54 that may be used to accept an insertion stylet (not shown) to facilitate lead implantation.

Further details describing the construction and method of manufacturing percutaneous stimulation leads are disclosed in U.S. patent application Ser. No. 11/689,918, entitled “Lead Assembly and Method of Making Same,” and U.S. patent application Ser. No. 11/565,547, entitled “Cylindrical Multi-Contact Electrode Lead for Neural Stimulation and Method of Making Same,” the disclosures of which are expressly incorporated herein by reference.

As will be described in further detail below, each of the neurostimulation leads 12 may include an actuating mechanism that is configured for modifying a linear shape of the neurostimulation lead 12 in order to move the electrodes 26 closer to a baseline position from which the neurostimulation lead 12 has laterally migrated.

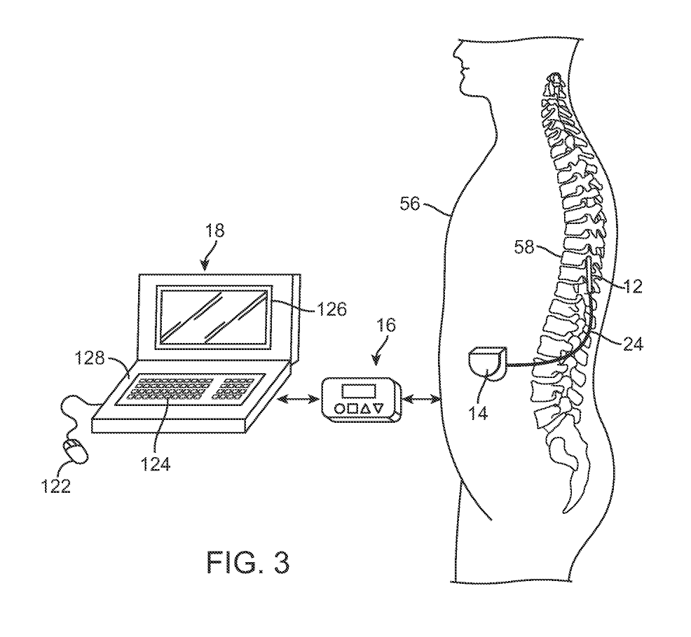

Referring to FIG. 3, the neurostimulation leads 12 are implanted at an initial position within the spinal column 58 of a patient 56. The preferred placement of the neurostimulation leads 12 is adjacent, i.e., resting near, or upon the dura, adjacent to the spinal cord area to be stimulated. Due to the lack of space near the location where the neurostimulation leads 12 exit the spinal column 58, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the neurostimulation leads 12. As there shown, the CP 18 communicates with the IPG 14 via the RC 16. While the neurostimulation leads 12 are illustrated as being implanted near the spinal cord area of a patient, the neurostimulation leads 12 may be implanted anywhere in the patient's body, including a peripheral region, such as a limb, or the brain. After implantation, the IPG 14 is used to provide the therapeutic stimulation under control of the patient. As previously mentioned in the background of the invention, either or both of the neurostimulation leads 12 may inadvertently migrate from their initially implanted position, either relative to each other or relative to a point in the tissue of the patient 56.

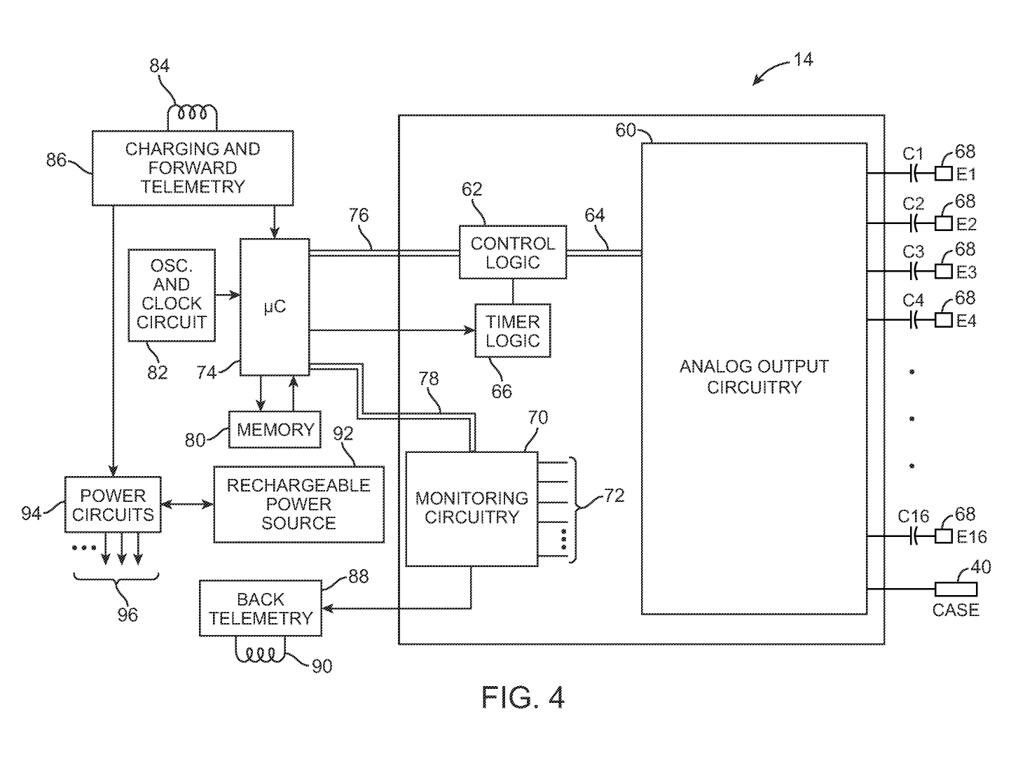

Turning next to FIG. 4, the main internal components of the IPG 14 will now be described. The IPG 14 includes stimulation output circuitry 60 configured for generating electrical stimulation energy in accordance with a defined pulsed waveform having a specified pulse amplitude, pulse rate, pulse width, pulse shape, and burst rate under control of control logic 62 over data bus 64. Control of the pulse rate and pulse width of the electrical waveform is facilitated by timer logic circuitry 66, which may have a suitable resolution, e.g., 10 μs. The stimulation energy generated by the stimulation output circuitry 60 is output via capacitors C1-C16 to electrical terminals 68 corresponding to the electrodes 26.

The analog output circuitry 60 may either comprise independently controlled current sources for providing stimulation pulses of a specified and known amperage to or from the electrical terminals 68, or independently controlled voltage sources for providing stimulation pulses of a specified and known voltage at the electrical terminals 68 or to multiplexed current or voltage sources that are then connected to the electrical terminals 68. The operation of this analog output circuitry, including alternative embodiments of suitable output circuitry for performing the same function of generating stimulation pulses of a prescribed amplitude and width, is described more fully in U.S. Pat. Nos. 6,516,227 and 6,993,384, which are expressly incorporated herein by reference.

The IPG 14 further comprises monitoring circuitry 70 for monitoring the status of various nodes or other points 72 throughout the IPG 14, e.g., power supply voltages, temperature, battery voltage, and the like. Notably, the electrodes 26 fit snugly within the epidural space of the spinal column, and because the tissue is conductive, electrical measurements can be taken from the electrodes 26. Significantly, the monitoring circuitry 70 is configured for taking such electrical measurements, so that, as will be described in further detail below, the positioning of each of the leads 12 relative to a reference point (e.g., the tissue and/or the other lead 12) may be determined. In the illustrated embodiment, the electrical measurements taken by the monitoring circuitry 70 for the purpose of determining the positioning of the leads 12 may be any suitable measurement, e.g., an electrical impedance, an electrical field potential, or an evoked potential measurement. The monitoring circuitry 70 may also measure impedance at each electrode 26 in order to determine the coupling efficiency between the respective electrode 26 and the tissue and/or to facilitate fault detection with respect to the connection between the electrodes 26 and the analog output circuitry 60 of the IPG 14.

Electrical data can be measured using any one of a variety means. For example, the electrical data measurements can be made on a sampled basis during a portion of the time while the electrical stimulus pulse is being applied to the tissue, or immediately subsequent to stimulation, as described in U.S. patent application Ser. No. 10/364,436, which has previously been incorporated herein by reference. Alternatively, the electrical data measurements can be made independently of the electrical stimulation pulses, such as described in U.S. Pat. Nos. 6,516,227 and 6,993,384, which are expressly incorporated herein by reference.

To facilitate determination of the positioning of each neurostimulation lead 12, electrical signals can be transmitted between electrodes carried by one of the neurostimulation lead 12 and one or more other electrodes (e.g., electrodes on the same neurostimulation lead 12, electrodes on the other neurostimulation lead 12, the case 40 of the IPG 12, or an electrode affixed to the tissue), and then electrical parameters can be measured in response to the transmission of the electrical signals. Alternatively, lead position can be monitoring using other means, such as strain gauge elements or optical fibers/coherence sensors within the leads 12. The position of the neurostimulation lead 12 relative to the tissue can then be determined based on the measured electrical parameters in a conventional manner, such as, e.g., any one or more of the manners disclosed in U.S. patent application Ser. No. 11/096,483, entitled “Apparatus and Methods for Detecting Migration of Neurostimulation Leads,” and U.S. patent application Ser. No. 12/495,442, entitled “System and Method for Compensating for Shifting of Neurostimulation Leads in a Patent,” which are expressly incorporated herein by reference. The position of the neurostimulation lead 12 relative to the other neurostimulation lead 12 can be determined based on the measured electrical parameters in a conventional manner, such as, e.g., any one or more of the manner disclosed in U.S. Pat. No. 6,993,384, entitled “Apparatus and Method for Determining the Relative Position and Orientation of Neurostimulation Leads,” U.S. patent application Ser. No. 12/550,136, entitled “Method and Apparatus for Determining Relative Positioning Between Neurostimulation Leads,” and U.S. patent application Ser. No. 12/623,976, entitled “Method and Apparatus for Determining Relative Positioning Between Neurostimulation Leads,” which are expressly incorporated herein by reference.

The IPG 14 further comprises processing circuitry in the form of a microcontroller 74 that controls the control logic 62 over data bus 76, and obtains status data from the monitoring circuitry 70 via data bus 78. The microcontroller 74 additionally controls the timer logic 66. The IPG 14 further comprises memory 80 and an oscillator and clock circuit 82 coupled to the microcontroller 74. The microcontroller 74, in combination with the memory 80 and oscillator and clock circuit 82, thus comprise a microprocessor system that carries out a program function in accordance with a suitable program stored in the memory 80. Alternatively, for some applications, the function provided by the microprocessor system may be carried out by a suitable state machine.

Thus, the microcontroller 74 generates the necessary control and status signals, which allow the microcontroller 74 to control the operation of the IPG 14 in accordance with a selected operating program and parameters. In controlling the operation of the IPG 14, the microcontroller 74 is able to individually generate electrical pulses at the electrodes 26 using the analog output circuitry 60, in combination with the control logic 62 and timer logic 66, thereby allowing each electrode 26 to be paired or grouped with other electrodes 26, including the monopolar case electrode, and to control the polarity, amplitude, rate, and pulse width through which the current stimulus pulses are provided.

The IPG 14 further comprises an alternating current (AC) receiving coil 84 for receiving programming data (e.g., the operating program and/or stimulation parameters) from the RC 16 in an appropriate modulated carrier signal, and charging and forward telemetry circuitry 86 for demodulating the carrier signal it receives through the AC receiving coil 84 to recover the programming data, which programming data is then stored within the memory 80, or within other memory elements (not shown) distributed throughout the IPG 14.

The IPG 14 further comprises back telemetry circuitry 88 and an alternating current (AC) transmission coil 90 for sending informational data (including the electrical parameter information, e.g., impedance data, field potential, and/or evoked potential measurements) sensed through the monitoring circuitry 70 to the RC 16. The back telemetry features of the IPG 14 also allow its status to be checked. For example, any changes made to the stimulation parameters are confirmed through back telemetry, thereby assuring that such changes have been correctly received and implemented within the IPG 14. Moreover, upon interrogation by the RC 16, all programmable settings stored within the IPG 14 may be uploaded to the RC 16.

The IPG 14 further comprises a rechargeable power source 92 and power circuits 94 for providing the operating power to the IPG 14. The rechargeable power source 92 may, e.g., comprise a lithium-ion or lithium-ion polymer battery. The rechargeable battery 92 provides an unregulated voltage to the power circuits 94. The power circuits 94, in turn, generate the various voltages 96, some of which are regulated and some of which are not, as needed by the various circuits located within the IPG 14. The rechargeable power source 92 is recharged using rectified AC power (or DC power converted from AC power through other means, e.g., efficient AC-to-DC converter circuits) received by the AC receiving coil 84. To recharge the power source 92, an external charger (not shown), which generates the AC magnetic field, is placed against, or otherwise adjacent, to the patient's skin over the implanted IPG 14. The AC magnetic field emitted by the external charger induces AC currents in the AC receiving coil 84. The charging and forward telemetry circuitry 86 rectifies the AC current to produce DC current, which is used to charge the power source 92. While the AC receiving coil 84 is described as being used for both wirelessly receiving communications (e.g., programming and control data) and charging energy from the external device, it should be appreciated that the AC receiving coil 84 can be arranged as a dedicated charging coil, while another coil, such as coil 90, can be used for bi-directional telemetry.

It should be noted that the diagram of FIG. 4 is functional only, and is not intended to be limiting. Those of skill in the art, given the descriptions presented herein, should be able to readily fashion numerous types of IPG circuits, or equivalent circuits, that carry out the functions indicated and described. It should be noted that rather than an IPG for the neurostimulator, the SCS system 10 may alternatively utilize an implantable receiver-stimulator (not shown) connected to the neurostimulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

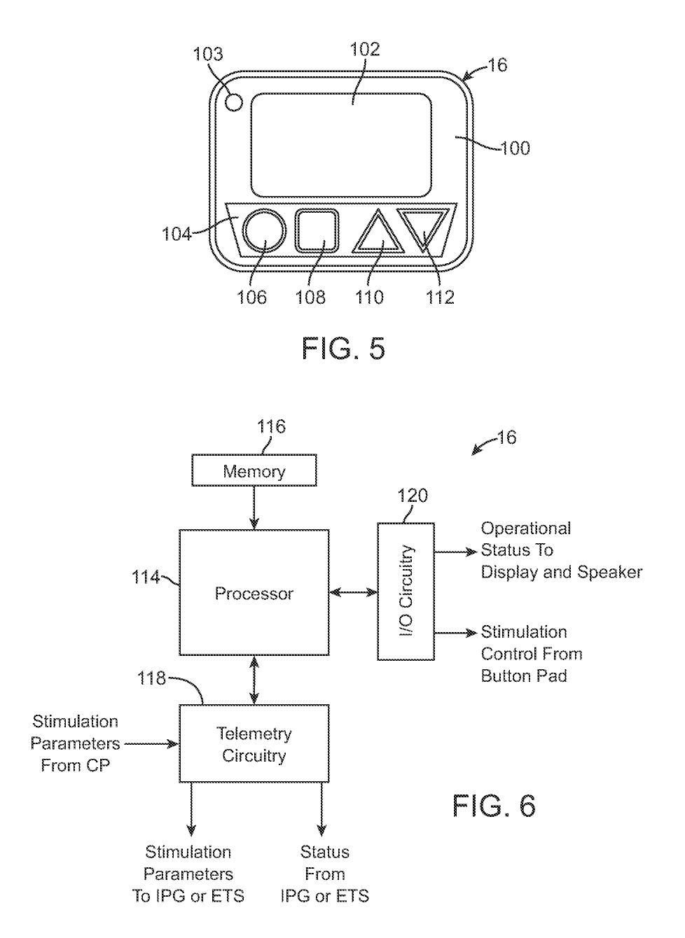

Referring now to FIG. 5, one exemplary embodiment of an RC 16 will now be described. As previously discussed, the RC 16 is capable of communicating with the IPG 14, CP 18, or ETS 20. The RC 16 comprises a casing 100, which houses internal componentry (including a printed circuit board (PCB)), a lighted display screen 102, an audio transducer (speaker) 103, and a button pad 104 carried by the exterior of the casing 100. In the illustrated embodiment, the display screen 102 is a lighted flat panel display screen, and the button pad 104 comprises a membrane switch with metal domes positioned over a flex circuit, and a keypad connector connected directly to a PCB. In an optional embodiment, the display screen 102 has touchscreen capabilities. The button pad 104 includes a multitude of buttons 106, 108, 110, and 112, which allow the IPG 14 to be turned ON and OFF, provide for the adjustment or setting of stimulation parameters within the IPG 14, and provide for selection between screens.

In the illustrated embodiment, the button 106 serves as an ON/OFF button that can be actuated to turn the IPG 14 ON and OFF. The button 108 serves as a select button that allows the RC 16 to switch between screen displays and/or parameters. The buttons 110 and 112 serve as up/down buttons that can be actuated to increase or decrease any of stimulation parameters of the pulse generated by the IPG 14, including pulse amplitude, pulse width, and pulse rate.

Referring to FIG. 6, the internal components of an exemplary RC 16 will now be described. The RC 16 generally includes a processor 114 (e.g., a microcontroller), memory 116 that stores an operating program for execution by the processor 114, and telemetry circuitry 118 for transmitting control data (including stimulation parameters and requests to provide status information) to the IPG 14 and receiving status information (including the measured electrical data) from the IPG 14 via link 34 (or link 32) (shown in FIG. 1), as well as receiving the control data from the CP 18 and transmitting the status data to the CP 18 via link 36 (shown in FIG. 1). The RC 16 further includes input/output circuitry 120 for receiving stimulation control signals from the button pad 104 and transmitting operational status information to the display screen 102 and speaker 103 (shown in FIG. 5). Further details of the functionality and internal componentry of the RC 16 are disclosed in U.S. Pat. No. 6,895,280, which has previously been incorporated herein by reference.

As briefly discussed above, the CP 18 greatly simplifies the programming of multiple electrode combinations, allowing the physician or clinician to readily determine the desired stimulation parameters to be programmed into the IPG 14, as well as the RC 16. Thus, modification of the stimulation parameters in the programmable memory of the IPG 14 after implantation is performed by a clinician using the CP 18, which can directly communicate with the IPG 14 or indirectly communicate with the IPG 14 via the RC 16. That is, the CP 18 can be used by the physician or clinician to modify operating parameters of the electrode array 26 near the spinal cord.

As shown in FIG. 3, the overall appearance of the CP 18 is that of a laptop personal computer (PC), and in fact, may be implemented using a PC that has been appropriately configured to include a directional-programming device and programmed to perform the functions described herein. Thus, the programming methodologies can be performed by executing software instructions contained within the CP 18. Alternatively, such programming methodologies can be performed using firmware or hardware. In any event, the CP 18 may actively control the characteristics of the electrical stimulation generated by the IPG 14 (or ETS 20) to allow the optimum stimulation parameters to be determined based on patient feedback and for subsequently programming the IPG 14 (or ETS 20) with the optimum stimulation parameters.

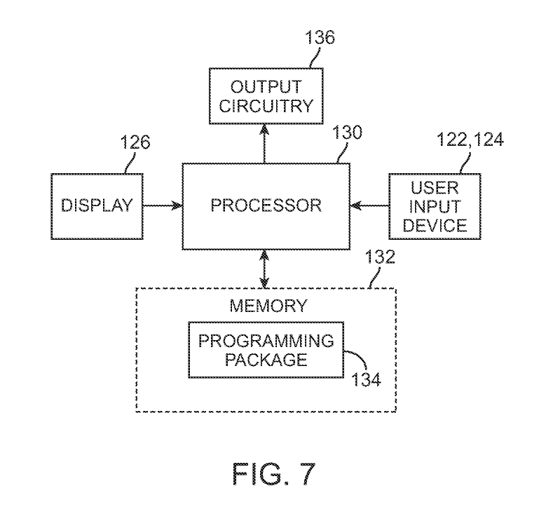

To allow the clinician to perform these functions, the CP 18 includes a mouse 122, a keyboard 124, and a programming display screen 126 housed in a case 128. It is to be understood that in addition to, or in lieu of, the mouse 122, other directional programming devices may be used, such as a joystick, or directional keys included as part of the keys associated with the keyboard 124. As shown in FIG. 7, the CP 18 generally includes a processor 130 (e.g., a central processor unit (CPU)) and memory 132 that stores a stimulation programming package 134, which can be executed by the processor 130 to allow a clinician to program the IPG 14 and RC 16. The CP 18 further includes telemetry circuitry 136 for downloading stimulation parameters to the RC 16 and uploading stimulation parameters already stored in the memory 116 of the RC 16 via link 36 (shown in FIG. 1). The telemetry circuitry 134 is also configured for transmitting the control data (including stimulation parameters and requests to provide status information) to the IPG 14 and receiving status information (including the measured electrical data) from the IPG 14 indirectly via the RC 16.

Significantly, the neurostimulation system 10 is capable of alerting the patient to the migration of each of the neurostimulation leads 12 from a baseline position. As discussed above, the position (whether it be the current position or the baseline position) is relative to a reference point, which can be, e.g., a point in the tissue of the patient, such that the patient is alerted to the absolute migration of respective neurostimulation lead 12, or can be, e.g., the position of the other neurostimulation lead 12, such that the patient is alerted to the migration of the respective neurostimulation lead 12 relative to each other. Preferably, the baseline position is the position at which the migrated neurostimulation lead 12 was in when the IPG 14 was initially programmed (e.g., the position that the neurostimulation lead 12 was initially implanted when the patient was initially fitted with the system 10) or reprogrammed (e.g., the position that the neurostimulation lead 12 was in when the patient subsequently returned to the clinician's office for adjustment of the stimulation parameters). That is, the baseline position is preferably the position of the neurostimulation lead 12 that is optimum for the stimulation parameters currently programmed into the IPG 14 and/or RC 16.

To this end, the neurostimulation system 10 determines the magnitude at which each neurostimulation lead 12 has migrated from its baseline position. In one embodiment, the neurostimulation system 10 accomplishes this function by determining a current position of the implanted neurostimulation lead 12 and computing a difference between the current position and the baseline position. As discussed above with respect to FIG. 4, the current position of the neurostimulation lead 12 can be determined by transmitting an electrical signal between one or more electrodes carried by the implanted neurostimulation lead 12 and one or more other electrodes, and measuring an electrical parameter (e.g., impedance, field potential, or evoked potential) in response to the transmission of the electrical signal.

After the magnitude at which each neurostimulation lead 12 has migrated from its baseline position is determined, the neurostimulation system 10 compares the determined magnitude to a threshold value representing an acceptable lead position tolerance, and outputs an alert signal to the patient based on this comparison, and in the illustrated embodiment, if the determined magnitude is equal to or exceeds the threshold value. The alert signal is user-discernible in that the patient can readily determine that at least one of the neurostimulation leads 12 has migrated relative to the baseline position outside of the acceptable lead position tolerance. Preferably, the alert signal is binary, meaning that it only indicates if a particular condition has been satisfied or not satisfied (i.e., at least one of the neurostimulation lead 12 has or has not migrated from the baseline position outside of the acceptable lead position tolerance).

In one embodiment, the RC 16 (or alternatively, the external charger 22) can alert the patient upon operative connection between the IPG 14 and the RC 16 (or alternatively, the external charger 22), e.g., upon establishing connection between the respective telemetry circuitries 88, 118 of the IPG 14 and RC 16. In this case, in addition to the alert function, the threshold value storage and processing functions are performed by the RC 16. In particular, and with reference back to FIGS. 5 and 6, the processor 114 determines the magnitude at which each of the neurostimulation leads 12 has migrated from its baseline position based on the measured electrical parameter data received by the IPG 14 via the telemetry circuitry 118, compares the determined magnitude to the threshold value recalled from the memory 116, and prompts an indicator (and in this case, the speaker 103) to output the alert signal in the form an aural signal (e.g., distinctive tones, patterns of sounds, music, voice messages, etc.) to the patient if the determined magnitude of migration is equal to or exceeds the threshold value.

Alternatively, the indicator can be the display 102, in which case, the outputted alert signal can take the form of a visual signal (e.g., a blinking icon). Or, the indicator be a mechanical transducer (not shown), in which case, the outputted alert signal can take the form of a vibratory signal (e.g., the case 100 can vibrate). Preferably, the processor 114 automatically prompts the indicator to output the alert signal immediately upon determination that the magnitude of migration is equal to or exceeds the threshold value, but alternatively, the processor 114 may prompt the indicator to output the alert signal only upon a user inquiry (e.g., pressing a button (not shown) on the RC 16) if the determined magnitude of migration is equal to or exceeds the threshold value.

In another embodiment, the IPG 14, itself, can alert the patient without establishing connection with the RC 16. In this case, in addition to the alert function, the threshold value storage and processing functions are performed by the IPG 14. In particular, and with reference back to FIG. 4, the microcontroller 74 determines the magnitude at which each of the neurostimulation leads 12 has migrated from its baseline position based on the measured electrical parameter data measured by the monitoring circuitry 70, compares the determined magnitude to the threshold value recalled from the memory 80, and prompts an indicator to output the alert signal to the patient if the determined magnitude of migration is equal to or exceeds the threshold value. Because the IPG 14 is implanted within the patient, the indicator may simply be the electrodes 26 on the neurostimulation leads 12, in which case, the outputted alert signal can take the form of a modulated neurostimulation signal (e.g., pulsing the neurostimulation signal on and off at a frequency less than the pulse frequency (e.g., every three seconds) or repeatedly increasing and decreasing the amplitude of the neurostimulation signal) that can be perceived by the patient as distinguished from normal, operative stimulation used for the therapy.

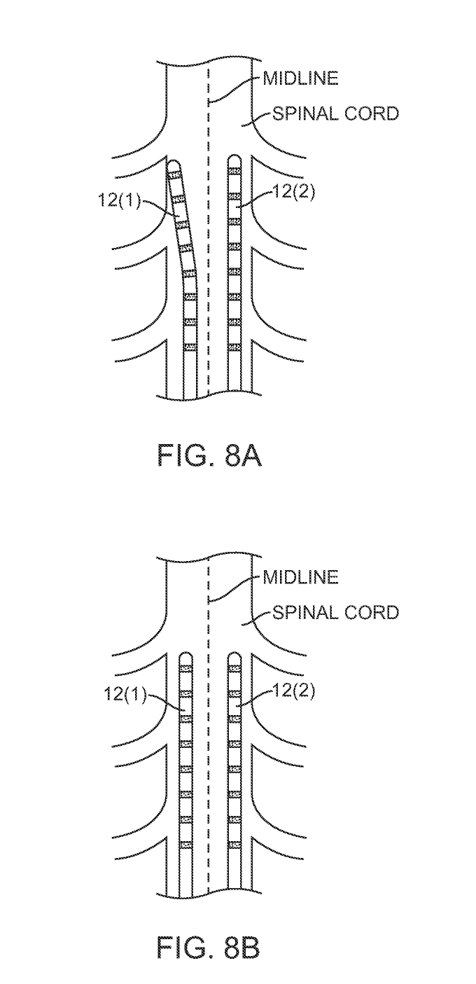

As briefly discussed above with respect to FIG. 2, each neurostimulation lead 12 may include an actuating mechanism (described in further detail below) that allows the linear shape of the respective neurostimulation lead 12 to be modified if it has migrated from its baseline position outside of the acceptable lead position tolerance. For example, if the neurostimulation lead 12(1) laterally migrates away from its baseline position (in this case, away from the midline of the spinal cord), as shown in FIG. 8A, the linear shape of the neurostimulation lead 12(1) may be modified, such that the distal end of the lead 12(1) is moved towards the midline of the spinal cord closer to the baseline position, as shown in FIG. 8B. This can be accomplished in a closed feedback loop manner by continuously or intermittently monitoring lead position and modifying the linear shape of the neurostimulation leads 12 in response to the monitored lead position without intervention by the user.

To this end, neurostimulation system 10 determines the magnitude at which each neurostimulation lead 12 has migrated from its baseline position, e.g., in the manner discussed above, compares the determined magnitude to a threshold value representing an acceptable lead position tolerance, and modifies the linear shape of the migrated neurostimulation lead 12 if the determined magnitude is equal to or exceeds the threshold value. Although the means for controlling the actuating device of each neurostimulation lead 12 is located in the IPG 14, the processor that performs the determination and comparison steps may be located in the IPG 14 or RC 16 (or alternatively, the external charger 22).

The actuating mechanism that can be operated to modify the linear shape of each neurostimulation lead can take the form of any one of a variety of mechanisms.

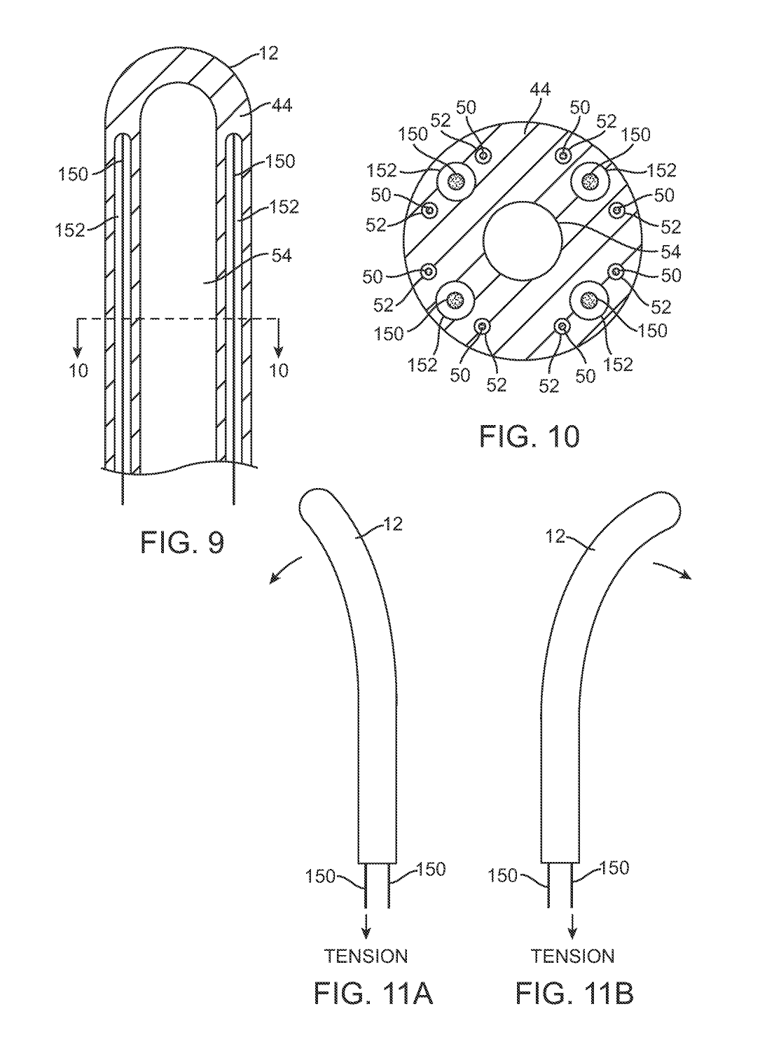

For example, and with reference to FIGS. 9 and 10, the actuating mechanism comprises a plurality of steering wires 150 (in this case, four steering wires) extending through individual lumens 152 within the lead body 44 of the respective neurostimulation lead 12. The distal end of each steering wire 150 is coupled to the distal end of the neurostimulation lead 12, such that tensioning of the steering wire 150 will deflect the distal end of the neurostimulation lead 12 in a particular direction. The proximal ends of the steering wires 150 may be terminated in a conventional steering mechanism (not shown) contained within the IPG 14.

The lumens 152 are circumferentially spaced apart by 90 degrees, such that tensioning of one of the steering wires 150 will deflect the distal end of the neurostimulation lead 12 in one of four different directions. However, once implanted, the distal end of the neurostimulation lead 12 will only need to be deflected in two opposite directions on a plane. For example, one of the steering wires 150 can be tensioned to deflect the distal end of the neurostimulation lead 12 to the left, as shown in FIG. 11A, and another of the steering wires 150 can be tensioned to deflect the distal end of the neurostimulation lead 12 to the right, as shown in FIGS. 11B. Thus, it can be appreciated that by applying a differential tension to the steering wires 150, the distal end of the neurostimulation lead 12 will deflect in a direction dictated by the steering wire 150 with the highest tension. In the case where the neurostimulation lead 12 laterally migrates away from the midline, the steering wire 150 that will move the distal end of the neurostimulation lead 12 back towards the midline can be tensioned while the remaining steering wires 150 remain relaxed.

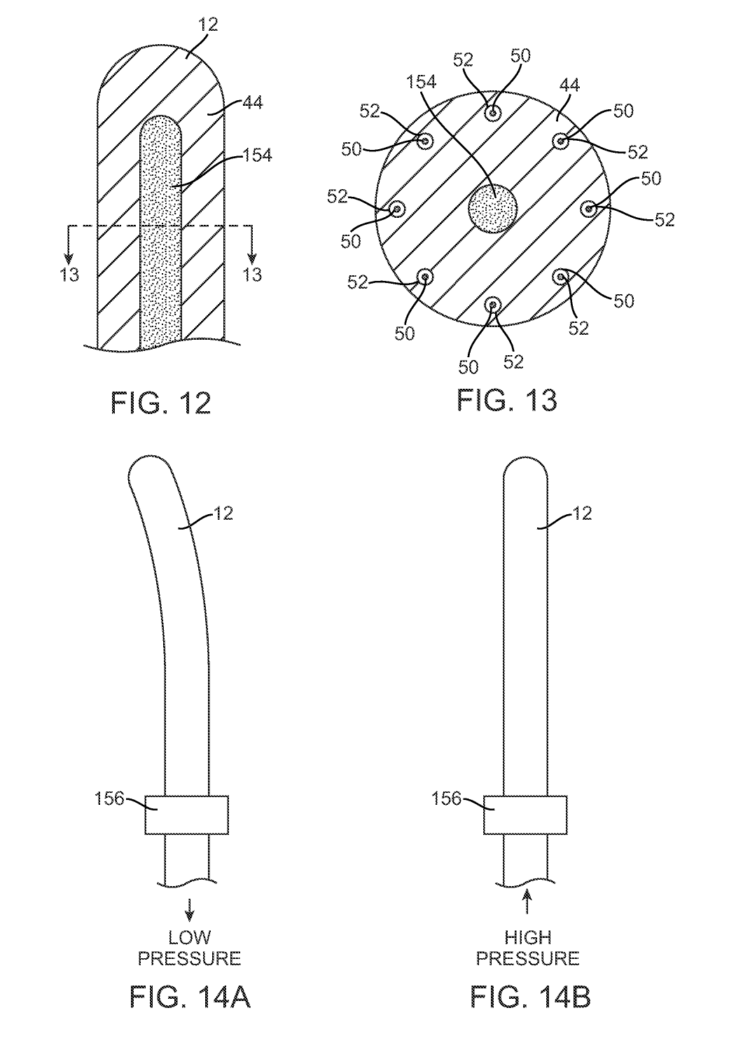

As another example, and with reference to FIGS. 12 and 13, the actuating mechanism comprises a fluid-filled (e.g., liquid or air) bladder 154 extending through the lead body 44 of the respective neurostimulation lead 12. The bladder 154 may double as a stylet lumen when delivering the neurostimulation lead 12 into the patient. The fluid-filled bladder 154 may contain any suitable medium in a liquid or gaseous state in which the pressure is easily adjustable. The pressure of the medium contained in the fluid-filled bladder 154 may be increased to straighten the distal end of the neurostimulation lead 12. In contrast, decreasing the pressure of the medium contained in the fluid-filled bladder 154 relaxes the distal end of the neurostimulation lead 12. In alternative embodiments, multiple fluid-filled bladders (not shown) can extend through the lead body 44. The proximal end of the bladder 154 may be terminated in a conventional pump mechanism (not shown) contained within the IPG 14.

Thus, it can be appreciated that by increasing the pressure within the fluid-filled neurostimulation lead 12, the distal end of the neurostimulation lead 12, when migrated away from the midline, as shown in FIG. 14A, will become rigid and thereby straighten to move it towards the midline, as shown in FIG. 14B. Preferably, an anchoring device 156, such as a suture sleeve, is used to fix the neurostimulation lead 12 to the tissue at a point proximal to the distal end of the neurostimulation lead 12, thereby preventing migration of the middle of the neurostimulation lead 12 away from the midline while allowing for mechanical leverage when straightening the distal end of the neurostimulation lead 12 to place the distal end of the neurostimulation 12 back in its baseline position.

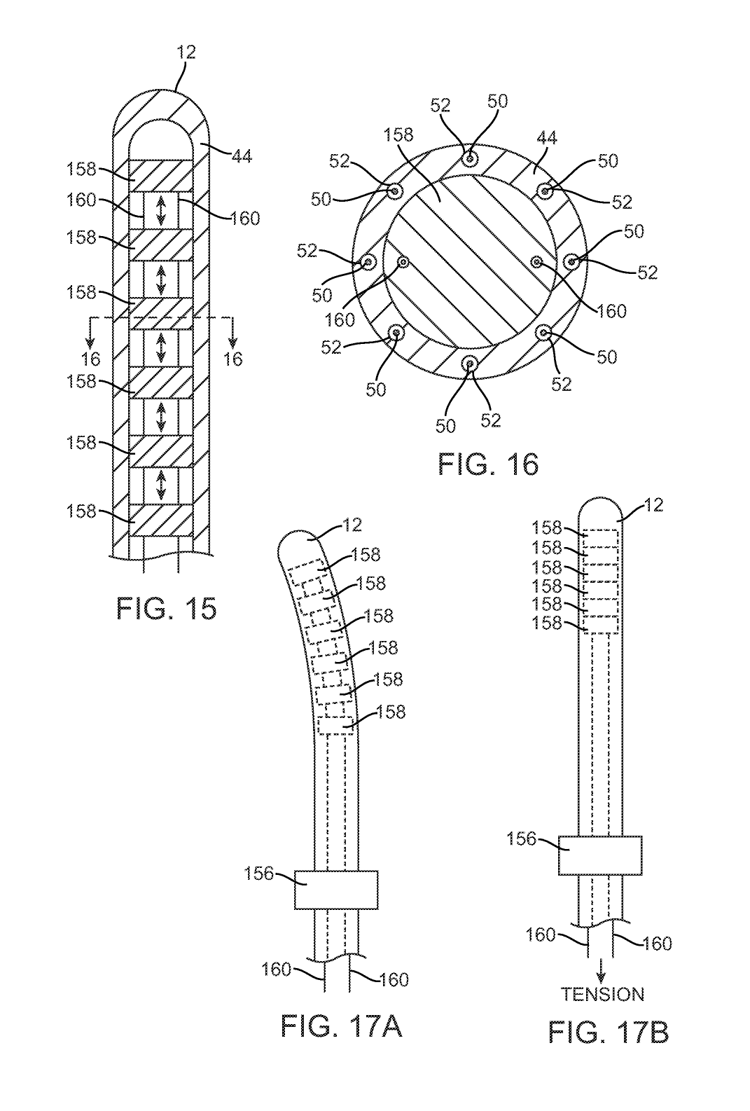

As still another example, and with reference to FIGS. 15 and 16, the actuating mechanism comprises a plurality of rigid cylindrical segments 158 extending through the lead body 44 of the respective neurostimulation lead 12. The cylindrical segments 158 can be displaced into contact with each other (as shown by the arrows) to straighten the distal end of the neurostimulation lead 12. In contrast, the cylindrical segments 158 can be displaced away (as shown by the arrows) from each other to relax the distal end of the neurostimulation lead 12. In the illustrated embodiment, pull wires 160 extend through the cylindrical segments 158, terminating in the distal-most cylindrical segment 158. Thus, tensioning the pull wires 160 will proximally displace the distal-most cylindrical segment 158, thereby forcing the cylindrical segments 158 into contact with each other and straightening the distal end of the neurostimulation lead 12. The proximal ends of the pull wires 160 may be terminated in a conventional wire tensioning mechanism (not shown) contained within the IPG 14. Alternatively, a proximal force can be applied by a mechanism (not shown) to the proximal-most cylindrical segment, thereby forcing the cylindrical segments 158 into contact with each other and straightening the distal end of the neurostimulation lead 12.

Thus, it can be appreciated that by forcing the cylindrical segments 158 into contact with each other, the distal end of the neurostimulation lead 12, when migrated away from the midline, as shown in FIG. 17A, will become rigid and thereby straighten to move it towards the midline, as shown in FIG. 17B. Preferably, the previously described anchoring device 156 used to fix the neurostimulation lead 12 to the tissue at a point proximal to the distal end of the neurostimulation lead 12, thereby preventing migration of the middle of the neurostimulation lead 12 away from the midline while allowing for mechanical leverage when straightening the distal end of the neurostimulation lead 12 to place the distal end of the neurostimulation 12 back in its baseline position.

Notably, combinations of different actuating mechanisms can be used, e.g., cylindrical segments can be used to straighten the neurostimulation lead 12, while wires can be used to steer the neurostimulation lead 12.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A neurostimulation system, comprising:
an implantable neurostimulation lead;
an implantable neurostimulator configured for delivering stimulation energy to the implantable neurostimulation lead;
an actuating device configured for modifying a linear shape of the neurostimulation lead after the implanted neurostimulation lead has migrated from a baseline position;
memory configured for storing a threshold value; and
at least one processor configured for determining a magnitude at which the neurostimulation lead has migrated from the baseline position, comparing the determined magnitude to the threshold value, and prompting the actuating device to modify the linear shape of the neurostimulation lead based on the comparison of the determined magnitude to the threshold value.

2. The neurostimulation system of claim 1, wherein the baseline position is a position of the neurostimulation lead relative to tissue.

3. The neurostimulation system of claim 1 wherein the baseline position is a position of the neurostimulation lead relative to another implantable neurostimulation lead.

4. The neurostimulation system of claim 1, wherein at least one processor is configured for determining the magnitude at which the implanted neurostimulation lead has migrated by determining a current position of the implanted neurostimulation lead and computing a difference between the current position and the baseline position.

5. The neurostimulation system of claim 4, wherein the neurostimulation is configured for transmitting an electrical signal between one or more electrodes carried by the implanted neurostimulation lead and one or more other electrodes, and measuring an electrical parameter in response to the transmission of the electrical signal, and wherein the at least one processor is configured for determining the current position of the implanted neurostimulation lead based on the measured electrical parameter.

6. The neurostimulation system of claim 1, wherein the threshold value represents an acceptable lead position tolerance.

7. The neurostimulation system of claim 1, wherein the actuating mechanism comprises at least one steering wire coupled to a distal end of the neurostimulation lead, wherein the at least one steering wire is configured for being tensioned to laterally deflect the distal end of the neurostimulation lead.

8. The neurostimulation system of claim 1, wherein the actuating mechanism comprises a fluid-filled bladder extending through the neurostimulation lead, wherein the pressure within the fluid-filled bladder is configured for being increased to straighten the distal end of the neurostimulation lead.

9. The neurostimulation system of claim 1, wherein the actuating mechanism comprises a plurality of a cylindrical segments spaced apart along the neurostimulation lead, wherein the cylindrical segments are configured to be displaced in contact with each other to straighten the distal end of the neurostimulation lead.

10. The neurostimulation system of claim 1, wherein the at least one processor is carried by the neurostimulator.

11. The neurostimulation system of claim 1, further comprising an external device carrying the at least one processor, wherein the at least one processor is configured for prompting the actuating device to modify the linear shape of the neurostimulation lead upon operative connection of the external device and the neurostimulator.

12. The neurostimulation system of claim 1, wherein the at least one processor is configured for prompting the actuating device to modify the linear shape of the neurostimulation lead only if the measured relative position is equal to or exceeds the threshold value.

13. The neurostimulation system of claim 12, wherein the at least one processor is configured for automatically prompting the actuating device to modify the linear shape of the neurostimulation lead if the measured relative position is equal to or exceeds the threshold value.

14. A method of correcting the migration of a neurostimulation lead implanted within a patient, the method comprising:
determining a magnitude at which the implanted neurostimulation lead has migrated from a baseline position;
comparing the determined magnitude to a threshold value; and
modifying the linear shape of the neurostimulation lead based on the comparison of the determined magnitude to the threshold value.

15. The method of claim 13, wherein the baseline position is the position at which the neurostimulation lead was initially implanted in the patient.

16. The method of claim 13, wherein the baseline position is a position of the implanted neurostimulation lead relative to tissue.

17. The method of claim 13, wherein the baseline position is a position of the implanted neurostimulation lead relative to another neurostimulation lead implanted within the patient.

18. The method of claim 13, wherein determining the magnitude at which the implanted neurostimulation lead has migrated comprises determining a current position of the implanted neurostimulation lead and computing a difference between the current position and the baseline position.

19. The method of claim 18, further comprising:
transmitting an electrical signal between one or more electrodes carried by the implanted neurostimulation lead and one or more other electrodes; and
measuring an electrical parameter in response to the transmission of the electrical signal;
wherein the current position of the implanted neurostimulation lead is determined based on the measured electrical parameter.

20. The method of claim 13, wherein the threshold value represents an acceptable lead position tolerance.

21. The method of claim 13, wherein the linear shape of the neurostimulation lead is modified by laterally deflecting the distal end of the neurostimulation lead.

22. The method of claim 13, wherein the linear shape of the neurostimulation lead is modified by straightening the distal end of the neurostimulation lead.

23. The method of claim 13, wherein the linear shape of the neurostimulation lead is modified upon operative connection between a neurostimulator connected to the neurostimulation lead and an external device.

24. The method of claim 13, wherein the linear shape of the neurostimulation lead is modified only if the measured relative position is equal to or exceeds the threshold value.

25. The method of claim 24, wherein the linear shape of the neurostimulation lead is automatically modified if the measured relative position is equal to or exceeds the threshold value.
